(19) United States
(12) Patent Application Publication
(10) Pub. No.: US 2024/0000730 A1
(43) Pub. Date: Jan. 4, 2024

(54) ANTIHISTAMINE SEMI-SOLID CHEWABLE GEL COMPOSITIONS AND METHODS OF MAKING AND USING THEREOF

(71) Applicant: Seattle Gummy Company, Renton, WA (US)

(72) Inventors: Feng WAN, Sammamish, WA (US); William Brenden Carlson, Shoreline, WA (US)

(73) Assignee: Seattle Gummy Company, Renton, WA (US)

(21) Appl. No.: 18/039,273

(22) PCT Filed: Dec. 1, 2021
(86) PCT No.: PCT/US2021/061470

§ 371 Date: May 29, 2023

(51) Int. Cl. A61K 31/138; A61K 9/00; A61K 31/4545; A61K 31/495; A61K 47/26; A61K 47/42; A61K 47/40; A61K 47/12; A61K 47/46; A61K 45/06; A61K 47/18

(52) U.S. Cl. CPC A61K 31/138; A61K 9/0056; A61K 31/4545; A61K 31/495; A61K 47/26; A61K 47/42; A61K 47/40; A61K 47/12; A61K 47/46; A61K 45/06; A61K 47/18

(57) ABSTRACT

An anti-histamine semi-solid chewable gel composition, comprising an active pharmaceutical ingredient (API) composition comprising an antihistamine, a gelling composition in a sufficient amount to provide a cohesive gelled product, a binding composition comprising a sugar, a sugar alcohol, and a water-soluble polymer stabilizer, wherein the polymer stabilizer comprises a polymer of monosaccharide monomers, and wherein the polymer comprises from about 5 to about 500 monosaccharide monomers, wherein the semi-solid chewable gel composition is substantially free of glucose, sucrose, and fructose.

### CROSS-REFERENCES TO RELATED APPLICATIONS

This application claims the benefit of priority from, and hereby incorporates by reference the entire disclosure, co-pending US Provisional applications for (1) Patent Ser. No. 63/119,657, filed Dec. 1, 2020; (2) Patent Ser. No. 63/119,661, filed Dec. 1, 2020; (3) Patent Ser. No. 63/119,658, filed Dec. 1, 2020; and (4) and Patent Ser. No. 63/119,660, filed Dec. 1, 2020.

### TECHNICAL FIELD

This application relates to semi-solid edible or chewable gel compositions with one or more bioactive such as antihistamines incorporated therein.

### BACKGROUND

Unless otherwise indicated herein, the materials described in this section are not prior art to the claims in this application and are not admitted being prior art by inclusion in this section.

Pharmaceuticals are available in a variety of dosage forms for treating the diseases. Dosages that are formulated to take orally including tablets, capsules, soft-gels, powders, chewable tablets, and liquid suspensions.

Tablets, capsules and soft-gels are difficult for individuals who have difficulties swallowing pills. This problem is magnified when the medications need to be taken 2-4 times per day to provide the desired therapeutic effect. Moreover, the need for a source of water or other liquid to assist with swallowing solid dosage forms can complicate administration.

Powders are often difficult to administer and chewable tablets can be hard to chew especially for seniors and young children. In addition, powders and chewable tablets often have an unpleasant after-taste.

Liquid suspensions or solutions are sometimes used as an alternative to solid oral dosage forms. However, the dosing with liquid dosage forms is not precise, which can lead to the administration of too little or too much medications. In addition, liquid dosage forms are messy and often have a bitter taste, which could impact person compliance.

Semi-solid chewable (gummy) composition could deliver medications and bioactive with an easier consumption profile. However, conventional gummy formulations are often packed with sugar and as a result having a high in glycemic index, making them unhealthy and potentially dangerous for diabetic patients.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides an antihistamine semi-solid chewable gel composition. In one embodiment, the antihistamine semi-solid chewable gel composition includes an active pharmaceutical ingredient (API) composition comprising an antihistamine, a gelling composition in a sufficient amount to provide a cohesive gelled product, a binding composition comprising a sugar, a sugar alcohol, or a combination thereof, and a water-soluble polymer stabilizer. The semi-solid chewable gel composition is substantially free of glucose, sucrose, and fructose. In one embodiment, the polymer stabilizer may be a polymer of monosaccharide monomers selected from glucose, fructose, mannose, galactose, arabinose, rhamnose, xylose, galacturonate, glucuronate, N-acetyl galactosamine, N-acetylglucosamine, or a combination thereof, and wherein the polymer comprises from about 5 to about 500 monosaccharide monomers. In one embodiment, the sugar may include L-fructose, L-glucose, L-galactose, allulose, sorbose, tagatose, D-maltose (1,4-diglucose), an isomer of D-sucrose (1,2-fructose glucose), trehalose, isomaltulose, raffinose or a combination thereof.

In one embodiment, the API composition comprises an H1-antihistamine. In one embodiment, the API composition comprises a H2-antihistamine. Example antihistamines include acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a derivative, salt or a combination thereof.

In one embodiment, the API composition comprises diphenhydramine, cetirizine, levocetirizine, loratadine, desloratadine, fexofenadine, azelastine, bilastine, rupatadine, or a derivative, salt or a combination thereof. In one embodiment, the API composition comprises cetirizine dihydrochloride or diphenhydramine dichloride.

In one embodiment, the API composition comprises essentially cetirizine or levocetirizine, or their salt thereof. Example salts may include hydrochloride salt, citrate, or a combination thereof. In one embodiment, the chewable composition comprises cetirizine at a concentration not less than about 0.01%, 0.05%, 0.1%, 0.15%, 0.2%, or 0.5% w/w. In one embodiment, cetirizine concentration is about 0.07% w/w. In one embodiment, cetirizine concentration is about 0.14% w/w. In one embodiment, cetirizine concentration is about 0.28% w/w. In one embodiment, the semi-solid chewable composition comprises from about 5 mg to about 40 mg cetirizine per dose. In one embodiment, the semi-solid chewable composition comprises about 2.5 mg, 5 mg, 10 mg, or 20 mg cetirizine per dose.

In one embodiment, the API composition comprises essentially diphenhydramine or bromodiphenhydramine. In one embodiment, the semi-solid chewable composition comprises diphenhydramine at a concentration not less than about 0.3%, 0.5%, or 1% w/w. In one embodiment, diphenhydramine concentration is about 0.35% w/w. In one embodiment, diphenhydramine concentration is about 0.71% w/w. In one embodiment, diphenhydramine concentration is about 1.42% w/w. In one embodiment, the semi-solid chewable composition comprises from about 2 mg to about 50 mg diphenhydramine per dose. In one embodiment, the semi-solid chewable composition comprises about 2 mg, 5 mg, 12.5 mg, 25 mg, or 50 mg diphenhydramine per dose.

In one embodiment, the API composition comprises essentially loratadine or desloratadine. In one embodiment, the semi-solid composition comprises loratadine at a concentration not less than about 0.05%, 0.1%, or 0.2% w/w. In one embodiment, loratadine concentration is about 0.07% w/w. In one embodiment, loratadine concentration is about 0.14% w/w. In one embodiment, loratadine concentration is about 0.28% w/w. In one embodiment, the semi-solid chewable composition comprises from about 2 mg to about 20 mg loratadine per dose. In one embodiment, the semi-solid chewable composition comprises about 2.5 mg, 5 mg, 10 mg or 20 mg loratadine per dose.

In one embodiment, the API composition comprises essentially fexofenadine. In one embodiment, the semi-solid composition comprises fexofenadine at a concentration not less than about 0.1%, 0.2%, 0.3%, 0.4%, 0.6%, 0.8%, 1%, or 1.2% w/w. In one embodiment, fexofenadine concentration is about 0.2% w/w. In one embodiment, fexofenadine concentration is about 0.4% w/w. In one embodiment, fexofenadine concentration is about 0.6% w/w. In one embodiment, the semi-solid chewable composition comprises from about 15 mg to about 180 mg fexofenadine per dose. In one embodiment, the semi-solid chewable composition comprises about 15 mg, 30 mg, 60 mg or 120 mg, 180 mg fexofenadine per dose.

The API composition may include a second API in addition to the antihistamine. In one embodiment, the API composition further comprises a H2 receptor blocker, a decongestant, a corticosteroid, a Leukotriene inhibitor, or a combination thereof. Example decongestant includes pseudoephedrine, oxymetazoline, tetrahydrozoline, a derivative or a combination thereof. Example corticosteroid includes budesonide, fluticasone furoate, fluticasone propionate, mometasone, triamcinolone, beclomethasone, ciclesonide, fluorometholone, loteprednol, prednisolone, prednisone, methylprednisolone, a derivative or a combination thereof. Example leukotriene inhibitor includes montelukast, a derivative thereof.

In one embodiment, the API composition may include cetirizine and pseudoephedrine or its derivative or salt thereof. In one embodiment, the API composition may include diphenhydramine and ibuprofen. In one embodiment, the API composition may include bromodiphenhydramine and codeine pseudoephedrine or its derivative or salt thereof. In one embodiment, the API composition may include diphenhydramine and pseudoephedrine or its derivative or salt thereof. In one embodiment, the API composition may include diphenhydramine and naproxen. In one embodiment, the API composition may include desloratadine and pseudoephedrine or its derivative or salt thereof. In one embodiment, the API composition may include fexofenadine and pseudoephedrine or its derivative or salt thereof.

In one embodiment, the semi-solid chewable gel composition may further comprise a complexing composition. The complexing composition can interact with antihistamine through coordinating, chelating, complexing, hydrogen-bonding, dipole-dipole interaction, van-der waals interaction, or a combination thereof and forming an antihistamine complex. In one embodiment, the antihistamine complex is capable of masking, lessening or reducing the antihistamine's taste, increasing antihistamine's solubility or stability in aqueous matrix, or a combination thereof. In one embodiment, the antihistamine complex is capable of masking and reducing the bitterness, astringent or metallic taste of the antihistamine. In one embodiment, the antihistamine complex is capable of increasing antihistamine's solubility in aqueous matrix therefore facilitating the incorporation of the antihistamine into the aqueous gummy matrix.

In one embodiment, the complexing composition comprises cyclodextrin, a nucleotide, resistant starch, or a combination thereof. In one embodiment, the complexing composition comprises protein, peptide, amide or polyamide, cluster dextrin, cyclodextrin, polydextrose, resistant starch, polyethylene glycol, polyunsaturated hydrocarbons, polyunsaturated fatty acids, mica, talc, zeolite, cellulose, plant particles, calcium carbonate, diatomaceous earth, chitosan, or a combination thereof.

In one embodiment, the complexing composition comprises an amide. Example amide includes without limitation 2-deoxy-2-aminoglucose N-acetyl, sialic acid N-acetyl, iminosugar N-acetyl, daunosamine N-acetyl, 2-deoxy-2aminogalactose N-acetyl, chitin, pectin, and amino acids.

Plant particles may be derived from various parts of a plant such as flower, fruit, seed, grain, nut, nutshell, root, leaves, or stems. In one embodiment, the plant particles comprise berry powder, nutshell powder, rice bran powder including without limitation strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, cranberry powder or blueberry powder. Nucleic acid rich plant is preferred such as strawberry, which is an octoploid.

In one embodiment, the complexing composition comprises cluster dextrin or cyclodextrin. In one embodiment, the cyclodextrin comprises alpha-dextrin, beta-cyclodextrin, gamma-cyclodextrin, or a combination thereof. In one embodiment, the cyclodextrin comprises essentially gamma-cyclodextrin. In one embodiment, the semi-solid pharmaceutical composition comprises antihistamine and cyclodextrin at a molar ratio of from about 1:1 to 1:100 or 1:1 to 1:20. In one embodiment, the molar ratio of antihistamine and cyclodextrin is about 1:5.

In one embodiment, the API composition comprises cetirizine, levocetirizine, diphenhydramine, loratadine, or fexofenadine, and the complexing composition comprises polyamide, cluster dextrin, cyclodextrin, or a combination thereof. In one embodiment, the API composition comprises cetirizine and the complexing composition comprises alpha-cyclodextrin. In one embodiment, the API composition comprises cetirizine and the complexing composition comprises beta-cyclodextrin. In one embodiment, the API composition comprises cetirizine and the complexing composition comprises gamma-cyclodextrin. In one embodiment, the API composition comprises diphenhydramine and the complexing composition comprises cyclodextrin, cluster dextrin, or a combination thereof. In one embodiment, the API composition comprises loratadine and the complexing composition comprises cyclodextrin.

In one embodiment, the binding composition comprises at least 2 or 3 binding agents selected from mannitol, maltitol, isomalt, erythritol, tagatose, allulose, sorbose, isomaltulose, trehalose, mannose, maltose, sorbitol, xylitol, ribose, xylose, tetroses, pentoses, hexoses, heptoses, their acid forms or a combination thereof.

In one embodiment, the binding composition comprises mannitol, maltitol, isomalt, or a combination thereof. In one embodiment, the binding composition comprises mannitol, sorbitol, or a combination thereof. In one embodiment, the binding composition comprises mannitol, sorbitol, erythritol or a combination thereof. In one embodiment, the binding composition comprises mannitol, sorbitol, isomalt, or a combination thereof. In one embodiment, the binding composition comprises mannitol, maltitol, sorbitol, or a combination thereof. In one embodiment, the binding composition comprises mannitol, maltitol, xylitol, or a combination thereof. In one embodiment, the binding composition comprises mannitol, xylitol, isomalt, or a combination thereof. In one embodiment, the binding composition comprises mannitol, xylitol, sorbitol or a combination thereof. In one embodiment, the binding composition comprises xylitol and erythriltol.

In one embodiment, the binding composition comprises mannitol, sorbitol, isomalt, resistant starch or a combination thereof. In one embodiment, the binding composition comprises mannitol, maltitol, sorbitol, maltodextrin or a combination thereof.

In one embodiment, the binding composition comprises tagatose, allulose, or a combination thereof. In one embodiment, the binding composition comprises tagatose, isomaltulose, or a combination thereof. In one embodiment, the binding composition comprises allulose, trehalose, isomaltulose, or a combination thereof. In one embodiment, the binding composition comprises maltitol, allulose, resistant starch or a combination thereof. In one embodiment, the binding composition comprises isomalt, allulose, resistant maltodextrin or a combination thereof.

In one embodiment, the binding composition comprises maltitol, allulose, or a combination thereof. In one embodiment, the binding composition comprises maltitol, sorbitol, allulose, or a combination thereof. In one embodiment, the binding composition comprises maltitol, tagatose, or a combination thereof. In one embodiment, the binding composition comprises allulose, tagatose, maltitol, isomalt, or a combination thereof. In one embodiment, the binding composition comprises isomalt, allulose, or a combination thereof. In one embodiment, the binding composition comprises isomalt, sorbitol, allulose, or a combination thereof. In one embodiment, the binding composition comprises allulose, xylitol, or a combination thereof. In one embodiment, the binding composition comprises allulose, maltitol, or a combination thereof.

The water-soluble polymer stabilizer may include a polysaccharide, a polyvinyl alcohol, a polyalcohol, a vinyl alcohol, a peptide, a cationic polymer, a polyphenol, or a combination thereof. Through significant and extensive experimentation, Applicant discovered that a stabilizing polymer significantly extend the stability of a low-sugar or sugar-free composition formulations for about 1.5, 2, 3, 4, 5, 7, 8, 9, or over 10 times when compared to same formulations without the polymer stabilizer. For example, the stability may be extended from about 2 weeks to about 8 months, about 3 months to about 9 months, about 6 months to about 12 months, about 5 months to about 14 months, about 9 months to over 24 months, about 10 months to over 36 months.

In one embodiment, the polymer stabilizer may be a polysaccharide. The polysaccharide may be cationic, anionic, or nonionic. It could be homo-polysaccharide or hetero-polysaccharide. Example polysaccharides include a polymer of glucose, fructose, mannose, galactose, arabinose, rhamnose, xylose, galacturonate, glucuronate, N-acetylgalactosamine, N-acetylglucosamine, or a combination thereof. In one embodiment, the polysaccharide comprises from about 5 to about 50 monomers.

In one embodiment, the polysaccharide comprises a polymer of glucose monomer or mannose monomer linked through glycosidic bonds. In one embodiment, the glycosidic bond is substantially free of 1,4-alpha-glycosidic bond. In one embodiment, the glycosidic bond comprises 1,2-alpha glycosidic bond, 1,3-alpha glycosidic bond, 1,2-beta glycosidic bond, 1,3-beta glycosidic bond, or a combination thereof.

In one embodiment, the polymer stabilizer comprises soluble fiber from tapioca, soluble corn fiber, soluble fiber from chicory root, soluble fiber from dandelion, maltodextrin, resistant maltodextrin, 6-20 β-1,4-linked glucopyranose units, 6-20 β-1,3-linked glucopyranose units, 6-20 β-1,2-linked glucopyranose units, 6-20 a-1,3-linked glucopyranose units, 6-20 a-1,2-linked glucopyranose units, or combination thereof. In one embodiment, the polymer stabilizer comprises maltodextrin.

In one embodiment, the polysaccharide comprises alpha-mannose monomers, beta-mannose monomers, beta-glucose monomers, or a combination thereof. In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccharide, fructo-oligosaccharide, galactomannan oligomers, oligomers of ribose, xylose, arabinose, rhamnose, or a combination thereof.

In one embodiment, the vinyl alcohol comprises a hydroxy methyl acrylate.

In one embodiment, the peptide comprises a collagen, a cationic peptide, or a combination thereof.

In one embodiment, the semi-solid chewable gel composition includes from about 1% to about 10%, about 1% to about 5%, about 2% to about 6%, about 0.5% to about 15% by weight of the polymer stabilizer.

In one embodiment, the ratio of the binding composition and the polymer stabilizer is from about 3:1 to about 20:1.

In one embodiment, the gelling composition comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

In one embodiment, the gelling composition comprises pectin. In one embodiment, the pectin has a methoxyl content (i.e., esterification degree or DE) not less than about 15%, 20%, 40%, 50% or 65%. In one embodiment, the methoxyl content is from about 15% to 40%, 15% to 25%, 16% to 24%, 30% to 70%, 50% to 65%, 55% to 65%, 59% to 63%, or 60% to 80%.

In one embodiment, the pectin has an amide content not less than about 15%, 20%, 30%, or 40%. In one embodiment, the amid content is from about 12% to 40%, 15% to 35%, 15% to 25%, 20% to 25%, 25% to 40%.

In one embodiment, the total of the methoxyl content and the amide content is from about 36% to 70%.

In one embodiment, the methoxyl content is more than about 25% and the amide content is not less than about 20. In one embodiment, the methoxyl content is from about 16% to 24% and the amide content is from about 20% to 25%. In one embodiment, the methoxyl content is from about 56% to 66% and the amide content is from about 0.1% to 0.5%.

In one embodiment, the semi-solid chewable gel composition may further comprise an herbal composition, an antioxidant composition, a vitamin composition, a mineral composition, an amino acid composition, a probiotics composition, or a prebiotics composition.

In one embodiment, the herbal composition comprises an adaptogen. In one embodiment, the herbal composition comprises one or more herbs having biological activity for alleviating or soothing allergy symptoms. In one embodiment, the herbal composition works with the antihistamine synergistically therefor is configured to enhance the antihistamine activity or increase the anti-allergy activity of the pharmaceutical composition. In one embodiment, the herbal composition may have the antihistamine activity. In one embodiment, the herbal composition comprises butterbur, quercetin, stinging nettles (Urtica dioica), bromelain, phleum pratense, tinospora cordifolia, European elderflower, sorrel, cowslip, verbena, gentian root, echinacea, grape seed, pycnogenol, pine bark extract, EPA, honey, cat's claw, albizzia (Albizzia lebbeck), baical skullcup (Scutellaria baicalensis), goldenseal, spirulina, bitter orange (citrus aurantium), lemon, eucalyptus, frankincense, Angelica sinensis, eyebright (Euphrasia officinalis), Gingko, milk thistle (Silybum marianum), red clover (Trifolium pratense), Yarrow (Achillea millefolium), rosemary, shiso, sage, peppermint, turmeric, curcumin, licorice, Astragalus, Ginseng, Artemisia argyi, Stephania tetrandra, coix seed, Citrus trifoliata, Citrus aurantium, Angelica dahurica, an extract, isolate or distillate thereof.

In one embodiment, the antioxidant composition comprises Vitamin E, Vitamin C, beta-carotene, gallic acid, selenium, selenium yeast, phenolics, anthocyanins, flavonoids, polyphenols, whey, bioflavonoids, theobromine, anthracenes, carotenoids, lutein, zeaxanthin, ginko biloba, berry extract, resveratrol, saffron, Sangre de grado (dragon's blood), cocoa, or derivatives thereof.

In one embodiment, the vitamin composition comprises vitamin A, B, C, D, E, K or a combination thereof.

The mineral composition may have the biological activity for alleviating or soothing allergy symptoms. In one embodiment, the mineral composition may work with the antihistamine synergistically therefor is configured to enhance the antihistamine activity or increase the anti-allergy activity of the pharmaceutical composition. In one embodiment, the mineral composition comprises salts of calcium, iron, zinc, magnesium, sodium, chloride, potassium, copper, molybdenum, manganese, phosphorus, iodine, nickel, or selenium, or a combination thereof. In one embodiment, the mineral composition consists essentially salts of zinc.

In one embodiment, the amino acid composition comprises one or more amino acids having biological activity for alleviating or soothing allergy symptoms. In one embodiment, the amino acid composition works with the antihistamine synergistically therefor is configured to enhance the antihistamine activity or increase the anti-allergy activity of the pharmaceutical composition. In one embodiment, the amino acid composition may have the antihistamine activity. In one embodiment, the amino acid composition comprises histidine, a branched chain amino acid, L-5 hydroxytryptophan (5-HTP), or its derivative thereof. In one embodiment, the amino acid composition comprises leucine, iso-leucine, valine, an essential amino acid, histidine, lysine, methionine, phenylalanine, threonine, tryptophan, L-theanine, beta-alanine, or its derivative thereof.

In one embodiment, the prebiotic composition comprises gum Arabic, chicory root powder or extract, wheat bran powder or extract, acacia gum, guar gum, Artichoke fiber, oat fiber, soluble corn fiber, inulin, resistant maltodextrin, resistant starch, or a combination thereof.

The probiotic composition comprises bifidobacteria, lactic acid bacteria, or a combination thereof. In one embodiment, the probiotic composition comprises Bifidobacterium lactis, Bifidobacterium longum, Lactobacillus acidophilus, Lactobacillus paracasei, Lactobacillus plantarum, Lactobacillus rhamnosus, Bacillus coagulans, Bifidobacterium bifidum, Lactobaccillus casei, Lactobaccillus gasseri, Lactobacillus salivarius, Lactobacillus bulgarius, or a combination thereof.

In one embodiment, the semi-solid chewable gel composition may further comprise an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the sweetener comprises xylitol, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylal-anine 1-methyl ester, salts thereof, licorice or its extracts or isolates, or a mixture thereof. In one embodiment, the semi-solid chewable gel composition comprises sucralose, aspartame, mongrosides, or a combination thereof. In one embodiment, the semi-sold chewable gel composition comprises sucralose. In one embodiment, the sucralose has a concentration from about 0.001% to about 0.1%, from about 0.005% to about 0.05%, from about 0.01 to about 0.05%, from about 0.02% to about 0.03% In one embodiment, the chewable gel composition is substantially free of artificial sweeteners or sugar substitutes.

In one embodiment, the antihistamine chewable gel composition may further include a coating composition. In one embodiment, the coating composition comprises isomalt, allulose, tagatose, xylitol, erythritol, sorbitol, mannitol, or a combination thereof. In one embodiment, the coating composition may have a particle size from about 0.6 mm to about 0.75 mm. In one embodiment, the coating composition may have a particle size of about 400 microns.

In one embodiment, the antihistamine chewable gel composition may include the binding composition comprising maltitol and at least one of allulose, xylitol, erythritol, maltitol, sorbitol, and mannitol, and the coating composition including maltitol.

In one embodiment, the chewable gel composition has a pH from about 3 to about 9. In one embodiment, the semi-solid chewable gel composition has a pH from about 2 to 4, 3 to 5, 2 to 5, 4 to 6, 5 to 7, or 6 to 9.

In one embodiment, the chewable gel composition comprises from about 0.5% to about 10% w/w of the gelling composition.

In one embodiment, the chewable gel composition comprises from about 60% to 80%, 65% to 75%, 65% to 60%, 68% to 69%, 67% to 71%, or about 70%, 67%, 68% w/w of the binding composition.

In one embodiment, the chewable composition comprises at least 2% w/w of the polymer stabilizer. In one embodiment, the semi-solid chewable gel composition comprises from about 3% to about 10%, from about 5% to about 8%, from about 6% to about 7% w/w of the polymer stabilizer.

In one embodiment, the chewable gel composition has a weight ratio of the binding composition and the polymer stabilizer from about 5:1 to 20:1, 6:1 to 15:1, 5:1 to 10:1, 10:1 to 12:1, 12:1 to 15:1, 10:1 to 20:1, or from 8:1 to 18:1. In one embodiment, the weight ratio is from about 10:1 to 15:1 In one embodiment, the antihistamine chewable gel composition may include cetirizine, levocetirizine, or a combination thereof, the complexing composition comprises cyclodextrine, wherein the antihistamine and the complexing composition have molar ratio from about 1:1 to about 1:3, the binding composition comprising maltitol, mannitol and isomalt, and the polymer stabilizer comprises polydextrose soluble fiber, wherein the binding composition and the polymer stabilizer have a w/w ratio from about 20:1 to 4:1, 10:1 to 5:1, 15:1 to 10:1, 8:1 to 4:1.

In one embodiment, the antihistamine chewable gel composition may include loratadine, or its salt or derivative thereof, the complexing composition comprises cyclodextrine, wherein the antihistamine composition and the complexing composition have molar ratio from about 1:1 to about 1:3, the binding composition comprising maltitol, mannitol and isomalt, the polymer stabilizer comprises polydextrose soluble fiber, wherein the binding composition and the polymer stabilizer have a w/w ratio from about 20:1 to 4:1, and the composition further comprises a sweetener comprising sucralose. In one embodiment, the sucralose has a concentration of from about 0.001% to 0.05%, 0.002% to 0.004%, 0.015% to 0.035%, or any concentration in between.

In another aspect, the application provides methods of making the semi-solid chewable gel composition.

In one embodiment, the method includes the steps of dividing the binding composition into a first binding portion and a second binding portion, combining a first mixture and water and heating to at a first elevated temperature to provide a first solution, wherein the first mixture comprises the first binding portion with the polymer stabilizer, combining the second mixture and water at a second elevated temperature to provide a second solution, wherein the second mixture comprises the gelling composition with a portion of binding composition that is equal to, twice, three time, or four times the mass of the gelling composition, the complexing composition, active ingredient, mixing the second solution into the first solution at a third elevated temperature to provide a third mixture, wherein the third mixture has a Brix number from about 80 to about 85 or from about 78 to about 86, adjusting pH of the third solution with a buffer salt to from about 3 to about 7.

In one embodiment, the method may further include the step of adding coloring agent, flavoring agent, or a combination thereof into the third mixture to provide a molding mixture having a Brix from about 78 to about 86. In one embodiment, the method may further include the step of adding the molding mixture to a preformed shaped cavity. In one embodiment, the method may further include the step of cooling the molding mixture (or mixture) in the preformed shaped cavity until the molding mixture forms into the semi-solid chewable gel composition piece.

The first, second and third elevated temperature may be independently from about 175° F. to about 275° F., from 175° F. to about 200° F., from 170° F. to about 210° F., or any temperature in between.

In one embodiment, the method comprises the following steps. In a first container, a gelling composition is mixed with a portion of a binding composition. Optionally, a buffering salt may be added. The components are mixed until homogeneous to provide a first mixture (Mix 1). In one embodiment, the gelling composition comprises pectin. In one embodiment, the portion of the binding composition comprises sorbitol and isomalt. In one embodiment, the buffering salt may include sodium citrate, potassium citrate, or a combination thereof.

In a second container, the remaining portion of the binding compositions added. In one embodiment, the remaining portion of the binding composition comprises sorbitol, isomalt, and mannitol. The components are mixed until homogeneous to provide a second mixture (Mix 2).

In a third container, a food acid is dissolved in an aqueous solution with additives such as coloring agent and flavoring agent. In one embodiment, the food acid comprises citric acid, malic acid, acetic acid, or a combination thereof. In one embodiment, the aqueous solution comprises water, ethanol, glycerol, or any combination thereof. All components are mixed and warmed until a homogenous solution is achieved to provide a third mixture (Mix 3). In one embodiment, the components are warmed to 175° F.

To a first reaction container, the active pharmaceutical Ingredient (API) composition is added.

Optionally, the complexing composition may be added. In one embodiment, the API composition comprises cetirizine, loratadine, diphenhydramine, or a derivative thereof. In one embodiment, the complexing composition comprises beta-cyclodextrin. Water is then added to the first reaction container.

In one embodiment, the water may be heated first before adding to the first reaction container. In one embodiment, the water is heated to at least 200° F.

Then the Mix 1 is added to the first reaction container with stirring to provide a first solution. The mixture is stirred until the gelling composition fully swells and disperses. In one embodiment, the first solution may be brought to a light boil.

To a second reaction container, Mix 2 is added, followed with addition of water. The components are mixed to provide a second solution. In one embodiment, the second solution may be brought to a boil.

Then, the first solution is combined with the second solution with mixing. The mixture is heated to a Brix number of at least 82 Brix. Then, Mix 3 is added dropwise with stirring to provide a molding mix having a Brix number of at least 82 Bix.

The modling mix was then added to a mold to provide individual gummy pieces. The gummy piece may be any shape and size. In one embodiment, the gummy piece may have a weight from about 2 g, 3 g, 4 g, 5 g, 6 g, 7 g, 7.5 g, to 8 g. The shape may be hexagon, square, half ball, gumdrop, heart, bear, or any other shapes.

The mold may be a silicon mold, a starch mold, or a sugar alcohol mold. In one embodiment, the i sugar alcohol mold is made by compacting sugar alcohol powder or particles in a container to create a compacted sugar alcohol mass, and stamping the compacted sugar alcohol mass with a desirable shape to create mold cavities in the compacted sugar alcohol mass. The molding mix may be injected or deposited into the mold cavities to form gummy pieces. In one embodiment, the sugar alcohol comprises maltitol, isomalt, or a combination thereof.

The gummy piece may be further coated with a coating composition. The coating composition may prevent gummy piece to stick with each other. The coating composition may include isomalt, maltitol, or other low glycemic sugar or sugar alcohol. In one embodiment, the coating composition comprises isomalt. In one embodiment, the coating composition comprises maltitol.

Through extensive experimentation, the Applicant discovered that the water solubility property of the coating composition is critical in maintaining gummy formulation stability. In one embodiment, the coating composition may have a water solubility of at least 2000 g/L, 1750 g/L, 1500 g/L, 1000 g/L, 500 g/L of water at room temperature. In one embodiment, the coating composition comprises isomalt, allulose, maltitol, maltodextrin, inulin, starch, bran, xylitol, sorbitol, tagatose, erythritol, or a combination thereof.

In another aspect, the application provides methods of treating a disease or modulating a physiological condition in a subject using the semi-solid chewable gel composition. In one embodiment, the allergy symptom is associated with seasonal allergic rhinitis, perennial allergic rhinitis, or chronic urticaria. In one embodiment, the allergy symptom comprises allergic rhinitis, sneezing, rhinorrhea, postnasal discharge, nasal pruritus, ocular pruritus, tearing, itchy eyes, urticaria, or a combination thereof. In one embodiment, the method includes the step of administrating an effective amount of the semi-solid chewable gel composition to the subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the ring structure of the cyclodextrin complexing with the cetirizine molecule.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the FIGURES, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

As used herein, “semi-solid chewable gel composition,” “chewable composition,” “gummy composition” and “gummy” are used interchangeably.

As used herein, “allulose” and “psicose” are used interchangeably.

As used herein, “isomaltulose” and “palatinose” are used interchangeably.

As used herein, “mix” and “mixture” may be used interchangeably.

### Active Pharmaceutical Ingredients (APIs)

Semi-solid formulation, such as gummy, can be used as an efficient delivery mechanism for an active pharmaceutical ingredient (API). A good tasting chewable composition would have several advantages over traditional pharmaceutical delivery formulations such as tablets, capsules and syrups. Unlike tablets and capsules, gummy can be taken with population with swallowing issues such as children and seniors. Unlike syrups, gummy can be accurately dosed. The chewable composition that is sweet and pleasing to consume leads to the advantage of increased patients' compliance in taking the medication. The act of chewing and dissolving the gummy allows for releasing of the API in the mouth and trans-mucosal absorption of the API. The rapid absorption of the API allows for the API to bypass the liver and avoid first-pass effect. Furthermore, trans-mucosal absorption allowed by the gummy gives faster symptom relief due to fast uptake of the API. Thus, solving the problems with gummy delivery of API's can lead to a superior technology for API delivery.

However, most of the APIs (such as cetirizine, diphenhydramine, etc.) have bitter, astringent, metallic or foul taste and are unpleasing. When placed into a traditional confectionary gummy formulation, the foul taste such as bitterness and metallic taste stays regardless of the quantity of sweeteners used. While a gummy delivery of APIs such antihistamines would be advantageous due to rapid absorption and symptom relief, simply incorporating these APIs into traditional gummy confectionary only lead to foul tasting undesirable products. Furthermore, gummy formulation is water based providing an aqueous matrix. Usually, a gummy formulation contains from about 12% to about 20% w/w of water content. APIs tend to instable in such aqueous environment. In addition, APIs are usually not water soluble make it technically impossible to disperse APIs homogenously into an aqueous gummy matrix to produce consistent products.

This application solved the above technical problems by providing semi-solid chewable composition formulations for the delivery APIs with pleasing taste and stability profile as well as homogenous products. In some embodiments, the application provides gummy pharmaceutical formulations that is excellent in texture, taste, and flavor, with proven solubility and stability of the APIs and allows for rapid delivery of antihistamines for fast relief of allergy symptoms.

The semi-solid chewable composition may include an API composition comprising an antihistamine. Example antihistamines may include acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, a derivative or salt thereof. The antihistamine composition may include one or more antihistamines.

In one embodiment, the API composition may include diphenhydramine, cetirizine, levocetirizine, loratadine, desloratadine, fexofenadine, azelastine, bilastine, rupatadine, or a derivative, salt or a combination thereof.

Both cetirizine and diphenhydramine used herein include all pharmaceutically useful derivative forms such as salts. In one embodiment, cetirizine may be cetirizine hydrochloride salt. In one embodiment, diphenhydramine may be diphenhydramine hydrochloride salt.

Cetirizine and diphenhydramine are indicated for symptom relief from allergies and colds. Cetirizine has a much lower indication of drowsiness side effect. In addition to relieving allergy symptoms, diphenhydramine can help with nausea and provide relief for motion sickness. The drowsiness causing effect makes diphenhydramine an effective sleep aid.

The chewable composition delivers from about 1 mg to about 100 mg of antihistamine APIs per dosage.

In one embodiment, the chewable composition delivers at least 2 mg cetirizine hydrochloride per dose. In one embodiment, the chewable composition delivers 2.5 mg, 5 mg, 10 mg, 20 mg, 40 mg, 80 mg, or 100 mg of cetirizine hydrochloride per dose.

In one embodiment, the chewable composition delivers at least 8 mg of diphenhydramine hydrochloride per dose. In one embodiment, the chewable composition delivers 2 mg, 4 mg, 8 mg, 10 mg, 12.5 mg, 25 mg, or 50 mg of diphenhydramine hydrochloride per dose.

In one embodiment, the chewable composition delivers at least 2 mg loratadine per dose. In one embodiment, the chewable composition delivers 2.5 mg, 5 mg, or 10 mg of loratadine per dose.

In one embodiment, the chewable composition delivers at least 15 mg fexofenadine per dose. In one embodiment, the chewable composition delivers 15 mg, 30 mg, 60 mg, 90 mg, or 180 mg of fexofenadine per dose.

The chewable composition may weight from about 2 g to about 10 g per dose. In one embodiment, the chewable composition weights about 2.5 g, 3 g, 3.5 g, 4 g, 5 g, 6 g, 6.5 g, 7 g, or 7.5 g per dose. The weight per dose may be any number in between the ranges.

### Complexing Compositions

Complexing compositions may be useful to complex with active ingredients such as herbal extract or active pharmaceutical ingredients therefore masking or modulating flavor profile or reducing bitterness. In one embodiment, the complexing composition comprises cyclic glucose.

Cluster dextrins have a ring structure with many branches of long chains of glucose units pendent to the ring. This has the effect of forming a helical structure. The helical structure along with the ring structure of cluster dextrins are both able to chelate small molecules such as the cetirizine molecule. The chelation takes place by the phenyl groups on cetirizine fitting inside the helical structure.

There are three major forms of cyclodextrin: alpha, beta, and gamma. Alpha cyclodextrin consists of a ring of 6 glucose units while beta has 7 glucose units in a ring and gamma has 8 glucose units in a ring. The ring structures form a crown. The inside of the crown is able to chelate small molecules.

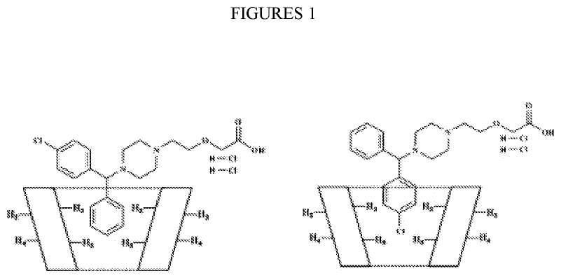

FIG. 1 shows the process of chelation of the cetirizine molecule. Either the phenyl group or the 4-chlorophenyl group fit inside the ring structure. The inside cavity of cyclodextrins is largely hydrophobic which is favorable for aromatic systems that are also hydrophobic. The formation of the chelate structure is endothermically favorable due to electrostatic interactions of the pi system of the aromatic moiety with in the hydrophobic cavity and electronic interaction with the hydrogen atoms and glycidyl ether bonds. It is these electronic interactions between these systems of the cyclodextrin and the pi system that gives the favorable heat of formation. The alpha, beta, and gamma cyclodextrins do not form the complex with cetirizine equally. Cetirizine forms more stable complex with β-cyclodextrin (K(a)=5641±358 M(−1)) than α-cyclodextrin (K(a)=1434±60 M(−1)). The association constants from ITC measurements for cetirizine-γ-cyclodextrin and cetirizine-α-cyclodextrin complexes were found to be 1200±50 and 1434±60 (−1) while the cetirizine-β-cyclodextrin complex was 5641±358 M(−1). Thus, the beta cyclodextrin has nearly four times the formation constant as the alpha and gamma cyclodextrin.

As shown in FIG. 1, the interior of the cylcodextrin is able to electronically interact with the phenyl groups of the cetirizine molecule. The phenyl group is a reverse quadrupole where the interior of the aromatic ring is very high in electron density and the exterior of the ring is electron deficient. The hydrogen atoms of the hydrophobic interior of the cyclodextrin is electronically attracted to the pi system of the aromatic ring. The hydrogen atoms of the aromatic ring are electronically attracted to the oxygen atoms of the cyclodextrin.

Diphenhydramine forms complexes with cyclodextrins. The mechanism is similar to that of cetirizine. Diphenhydramine forms the more stable complex with β-cyclodextrin (K(a)=4988 M⁻¹) than either α-cyclodextrin and gamma-cyclodextrin which are both K(a)=1000 M(−1). In one embodiment, the complexing composition comprises beta cyclodextrin, which is configured to form antihistamine complex. In some embodiments, the complexing composition comprises alpha-cyclodextrin, gamma-cyclodextrin, or a combination there. The action of coordination with diphenhydramine is similar to that of cetirizine.

Cluster dextrin also coordination with cetirizine or diphenhydramine. Cluster dextrins have a broad range of cyclic ring and helical structures. Statistically some cyclic and helical structures meet the criteria for complexing with cetirizine or diphenhydramine. Without being limited by theory, the mechanism of complexing between cetirizine or diphenhydramine and cluster dextrin molecules is the same electronic interactions that occur as the alpha, beta, and gamma cyclodextrins.

Through extensive research and experimentation, Application discovered that an effective method of flavor mitigation for an API that has functionality similar in structure to nucleotide bases is the addition DNA or RNA or sources rich in RNA and DNA. A chemical structure such as Famciclovir which has a guanosine structural unit will be naturally attracted and bind the cytosine bases in DNA thereby chelating the API and reducing its contribution to the overall flavor of the gummy.

Foods that are particularly high in DNA are the fruits and vegetables and their powders. Fruits and vegetables have many strands of DNA and RNA that go beyond the normal helix (diploid). The strawberry is famous for being particularly rich in DNA. Each cell in a strawberry contains 8 copies of its genetic information (octoploid), while most animal cells only contain 2 copies (diploid). Other examples of high-density DNA and/or RNA are the sweet potato (hexaploid), sugar cane (octoploid), apple (triploid), peanut (tetraploid), and kumquats (tetraploid). Any polyploidy plant material can be used as the DNA/RNA source.

### Gelling Composition

In one embodiment, the gelling composition comprises pectin, gelatin, or a combination thereof. In one embodiment, gelatin may be combined with other hydrocolloids—pectin, agar, starch, gum Arabic—to create desired textures. In one embodiment, gelatin may be combined with gum arabic as the gelling composition.

In one embodiment, the gelling composition comprises starch such as amylose starch or modified starch.

In one embodiment, the gelling composition comprises agar. Agar may be combined with locust bean gum as a gelling composition. Locust bean gum helps to prevent weeping of agar gels. The two polysaccharides from agar and locust bean gum synergistically interact with each other to form a strong gel that does not weep.

In one embodiment, the gelling composition comprises carageenans. Carrageenans or carrageenins are linear sulfated polysaccharides. In one embodiment, locust bean gum may be used with kappa-carrageenan to prevent weeping. Gels formed from kappa-carrageenan and potassium ions are thermally reversible.

In one embodiment, the gelling composition comprises alginic acid or alginate. Alginate may form strong hydrogels when crosslinked with calcium ions.

### Binding Composition

Binding composition binds the semi-solid chewable gel (gummy) together through interaction with the gelling composition. The interaction may be through hydrogen bonding or through covalent bonding. In one embodiment, the binding composition comprises sugars, sugar derivatives, sugar alcohols, or a combination thereof. The binding composition may keep texture of the product soft by acting as a humectant.

Conventional gummy formulation typically contains 60-85% w/w sugars, leading to high glycemic index (GI) formulation that is not safe for diabetic population. Moreover, conventional sugars such as glucose, sucrose, and fructose are cariogenic and high in both glycemic index and calorie. Thus, for many situations, it would be advantageous to have a sugar free gummy or a low GI gummy formulation as disclosed thereof. In one embodiment, the disclosed composition that is free of sucrose, glucose and fructose.

In one embodiment, the binding composition comprises a mono- or di-saccharide (i.e., sugar) having a glycemic index of less than 80, 35, 30, 25, 20, 15, or 10, a sugar alcohol, or a combination thereof. In one embodiment, the semi-solid chewable composition is substantially free of sugar having a glycemic index of more than 35. In one embodiment, the semi-solid chewable composition has a glycemic index of not more than 8, 10, 15 or 20.

In one embodiment, the binding composition comprises a low GI sugar having a glycemic index (GI) of not more than 18, 20, 30, 35, or 70. In one embodiment, the low GI sugar comprises allulose, sorbose, tagatose, trehalose, isomaltulose, raffinose, or a combination thereof. In one embodiment, the binding composition comprising tagatose, allulose, sorbose, isomaltulose, trehalose (also known as mycose), mannose, maltose, ribose, xylose, tetroses, pentoses, hexoses, heptoses, their acid forms or a combination thereof. In one embodiment, the binding composition consists essentially of allulose, isomaltulose, and a third low GI sugar selected from a group consisting of trehalose, sorbose, tagatose, or a combination thereof.

Through extensive experimentation, processes are developed by Applicant to used allulose, sorbose, tagatose, isomaltulose, trehalose in the gummy formulation allowing these sugars behave like conventional sugars such as sucrose and fructose but without the caloric significance of the convention sugars.

In one embodiment, the binding composition comprises allulose, trehalose and isomaltulose. In one embodiment, the binding composition comprises more than 20% isomaltulose. In one embodiment, the binding composition comprises from 15% to 35% isomaltulose. In one embodiment, the binding composition comprises not more than 75% of allulose. In one embodiment, the binding composition comprises from 45% to 60% allulose. In one embodiment, the binding composition comprises not more than 45% of trehalose.

In one embodiment, the binding composition comprises allulose and tagatose. In one embodiment, the binding composition comprises not more than 50% tagatose. In one embodiment, the binding composition comprises from 30% to 45% tagatose. In one embodiment, the binding composition comprises not more than 70% of allulose.

In one embodiment, the binding composition comprises isomaltulose and tagatose. In one embodiment, the binding composition comprises from 30% to 60% tagatose. In one embodiment, the binding composition comprises not more than 70% of isomaltulose.

In one embodiment, the binding composition comprises essentially of sugar alcohols. Example sugar alcohols include sorbitol, mannitol, erythritol, xylitol, isomalt, maltitol, lactitol, and hydrogenated starch hydrolysates. In one embodiment, the binding composition comprises mannitol, maltitol, or isomalt. In one embodiment, the binding composition comprises mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof. In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof. In one embodiment, the binding composition consists essentially of mannitol, sorbitol, or a combination thereof. In one embodiment, the binding composition consists essentially of mannitol, sorbitol, erythritol or a combination thereof.

Mannitol has 50-70% of the relative sweetness of sugar. Mannitol lingers in the intestines for a long time and therefore may cause gastric distress.

Erythritol ((2R,3S)-Butan-1,2,3,4-tetrol) is a non-caloric polyol with a moderate sweetness of 60-80% of sucrose. Erythritol can improve the mouth feeling and mask certain unwanted aftertastes such as astringency and the irritant effect of intense sweeteners.

Sorbitol has 50% of the relative sweetness of sugar. It has less of a tendency to cause gastric distress than mannitol. Sorbitol is highly soluble in water and is an excellent humectant. Xylitol is also called “wood sugar” has the same relative sweetness as sugar. Xylitol not only non-cariogenic, it actually prevents tooth decay. Lactitol has about 30-40% of sugar's sweetening power. Its taste and solubility profile resemble sugar. Isomalt is 45-65% as sweet as sugar and does not tend to lose its sweetness or break down during the heating process. Isomalt absorbs little water. Maltitol is 75% as sweet as sugar and provides about 2-3 kcal/g. It gives a creamy texture to the formulation.

In one embodiment, the binding composition may include hydrogenated starch hydrolysates (HSH).

### Polymer Stabilizer

Through extensive research, the applicant discovered that the semi-solid chewable gel compositions comprising essentially sugar alcohol or low glycemic sugars as binding agents are prone to crystallization leading to insatiability of the formulation. The applicant further discovered that certain concentration of polymer stabilizers may be used to promote the thermodynamic stability of the formulation and reducing the crystallization. In one embodiment, the semi-solid chewable gel composition is substantially free of glucose, sucrose, and fructose and further comprises a polymer stabilizer. In one embodiment, the polysaccharide may be water soluble.

In one embodiment, the polymer stabilizer comprises a polysaccharide of mono- or di-saccharide monomers. In one embodiment, the monomers may include glucose, fructose, mannose, galactose, arabinose, rhamnose, xylose, galacturonate, glucuronate, N-acetylgalactosamine, N-acetylglucosamine, or a combination thereof. The polysaccharide comprises from about 5 to about 50 monosaccharide monomers.

In one embodiment, the polymer stabilizer may include a mushroom polysaccharide. In one embodiment, the mushroom polysaccharide may derive from schizophyllum commune (Schizophyllum commue), Brazilian mushroom (Agarics blaze), Cordyceps sinensis (Cordyceps sinensis), glossy ganoderma (Ganoderma lucidum), rainbow conk (Coriolus versicolor), camphor tree sesame (Anthodia camphorate), Phellinus (Phellinus linteus), coral mushroom (Pleurotus citrinopileatus), mushroom (Lentinula edodes), Liu Songgu (Agrocybe aegerita), Hericium erinaceus (Hericium erinaceus), pleurotus eryngii (Pleurotus eryngiig), petal fine and soft (Sparrasis crispa), black fungus (Auricularia auricula), Asparagus (Flammulina velutipes) or a combination thereof. In one embodiment, the mushroom polysaccharide may include chitin, hemicellulose, α- and β-glucans, mannans, xylansand, or galactans. In one embodiment, the mushroom polysaccharides may include β-glucan polymers, with the main chain consisting of β-(1→3) linkages with some β-(1→6) branches as well as chitin, mannans, galactans, and xylans.

In one embodiment, the polymer stabilizer may include a polysaccharide derived from an herb. In one embodiment, the polysaccharide may derive from Cistanche deserticola, Astragalus membranaceus, Rubia cordifolia, Nerium indicum, Adhatoda vasica, Withania sonnifera, and Glycyrrhiza glabra, aloe vera, Bletilla striata, Konjac, Goji berry, elderberry, or a combination thereof.

In one embodiment, the semi-solid composition may include from about 5% to about 15% of polymer stabilizer.

In one embodiment, the ratio of the binding composition and the polymer stabilizer is from about 8:1 to about 20:1.

### Coating Composition

The gummy pieces disclosed herein may be coated with a coating composition. In one embodiment, the coating composition may include matitol, isomalte, allulose, or a combination herein.

In one embodiment, the coating compositions may include sugar, sugar alcohol, or a combination thereof. Example sugars may include allulose, sorbose, tagatose, trehalose, and isomaltulose, or a combination thereof. Example sugar alcohols may include erythritol, sorbitol, mannitol, maltitol, isomalt, xylitol, or a combination thereof. In one embodiment, the coating composition comprises resistant starches, fibers, inulin, or a combination thereof.

The semi-solid chewable gel composition may further comprise an active pharmaceutical ingredient, an herbal composition, an antioxidant composition, a vitamin composition, a mineral composition, an amino acid composition, a probiotics composition, or a prebiotics composition.

### Herbal Composition

Herbal extract or derivatives useful for the application may include flavanoids, allied phenolic compounds, polyphenolic compounds, terpenoids, alkaloids, sulphur-containing compounds, polysaccharides, flavone, flavonoids, quinone, or combinations thereof. In one embodiment, the herbal composition may comprise an adaptagen.

### Antioxidants

Antioxidant may include astaxanthin, carotenoids, coenzyme Q10 (“CoQ10”), flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof.

### Vitamins

Vitamins may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D, vitamin E, vitamin K, K1 and K2 (i.e., MK-4, MK-7), folic acid, biotin, choline, or combinations thereof.

### Minerals

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

### Amino Acids

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, camitine, its derivative, or combinations thereof. In one embodiment, the amino acid may be a branched-chain amino acid. In one embodiment, the amino acid may be a natural amino acid. In one embodiment, the amino acid may be a non-natural amino acid.

Taurine, or 2-aminoethanesulfonic acid, is an amino acid that is widely distributed in animal tissues. It is a major constituent of bile and can be found in the large intestine, and accounts for up to 0.1% of total human body weight. Taurine can reduce bitterness by 50% when added at a concentration of 300 mM.

### Probiotics

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects.

Probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In one embodiment, the chewable composition contains probiotic strains in an amount ranging from 10⁵ and 10¹² cfu/g or 10⁷-10¹⁰ cfu/g.

### Prebiotics

Useful prebiotics are Lactose, Inulin, Fructo oligosacccharides, Galacto oligosaccharides and Xylo oligosaccharides. Prebiotics are naturally found plenty in certain fruits like bananas, asparagus, garlic, tomato and onion wheat. The characteristic features of ideal prebiotics are as follows. They are neither to be hydrolysed nor absorbed by mammalian enzymes or tissues. They are selectively enriched with a limited number of beneficial bacteria. The most important characteristic feature is that prebiotics can alter the intestinal micro-flora and its activities. Prebiotics can also change luminal or systemic aspects of the host defense system.

Prebiotics when combined with probiotics have many advantages. Basically, prebiotics selectively stimulate the growth of probiotics, which is dose and strain dependent. Prebiotics serve as a selective growth substrate for the probiotics strain during fermentation, during the period of storage, or during its passage through the gut.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

### Humectants

A pharmaceutically acceptable humectant can include one or a mixture of humectants, such as, for example, glycerin, sorbitol and polyethylene glycol, for the gummy composition embodiments. The humectant content can be in the range of from about of 1% w/w to about 30% w/w and such as about 2% w/w to about 25% w/w.

Humectants are low molecular weight species that give the sensation of moisture in the gummy formulation. The humectants mimic water in the gummy formulation and which allows for very low water levels (2-6%) in the gummy formulation. The humectant can act as a moisturizing agent in the mouth.

The humectant prevents the gummy formulation from drying out and helps to maintain softness and shelf life. A very low vapor press is an important property of the humectant for the maintenance of softens so it does not evaporate out. Example humectants are glycerol (glycerin, 1,2,3-propantriol), propylene glycol (1,2-propandiol), aloe vera gel, glyceryl triacetate, and a-hydroxyacids (lactic acid).

### Sweeteners

Example sugar substitutes include saccharin, aspartame, sucralose and acesulfame K In one embodiment, the sweetener is selected from one or more of saccharin, aspartame, cyclamate, sucralose, Stevia, mannitol, sorbitol, xylitol and similar glycols.

### Plasticizers

Plasticizers impart flexibility by lowering the glass transition temperature of the polymers. They act in gummy formulation decrease brittleness and increase chewiness. Some plasticizers that can be added to the chewable are lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium and potassium stearates.

### Flavoring Agents

A pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, such as spearmint essential oil. Some flavoring agents can also act as sweeteners and can be use as such and include, for example, neohespiridin dehydrochalcone, xylitol, sucralose, and mixtures thereof, such as xylitol. The flavoring agent can be in the range of from about of 0.05% w/w to about 3% w/w and such as about 0.5% w/w to about 1% w/w.

Flavors and colors are for sensory appeal. Flavor components in gum exist in liquid, powder or micro-encapsulated forms. Liquid flavor incorporations are either water-soluble, oil-soluble, or water-dispersible emulsions. The oil-soluble flavors remain in the gum longer, resulting in longer lasting flavor sensations, because the gum base is hydrophobic and attracted to oil-based components. Conversely, water soluble flavors dissolve into saliva and as such are extract out of the gum which leads to quicker loss in flavor.

In one embodiment, the flavoring agent is a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

### Flavoring Modulating Agents

The gummy pharmaceutical composition may further include flavoring modulating agent. In one embodiment, the flavor modulating agent comprises mannitol. Mannitol is a sugar-alcohol. It is derived from the aldose called mannose. Mannitol can help mask bitterness. Mannitol masks bitterness by a mechanism that involves the endothermic nature of mannitol dissolving into water.

In one embodiment, the flavoring modulating agent comprises taurine. Taurine, or 2-aminoethanesulfonic acid, is an organic compound that is widely distributed in animal tissues. Taurine can reduce bitterness by 50% when added at a concentration of 300 mM.

### Surfactants

A pharmaceutically acceptable surfactant can include one or a mixture of surfactants, such as, for example, certain nonionic, anionic and amphoteric surfactants and can include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof including, for example, sodium lauryl sulfate (SLS) or sodium lauryl ether sulfate (SLES) as well as anionic taurate surfactants including, for example, sodium methyl cocoyl taurate and sodium methyl oleoyl taurate, PEG caster oils, and PEG hydrogenated caster oils, including, for example, PEG-60 Hydrogenated Castor Oil. In one embodiment, the chewable composition includes sodium lauryl sulfate (SLS) and sodium methyl cocoyl taurate, a mixture of sodium lauryl sulfate (SLS) and sodium methyl cocoyl taurate. The surfactant content can be in the range of from about of 0.1% w/w to about 10% w/w and such as about 2% w/w to about 5% w/w.

In one embodiment, the surfactant is selected from one or more of sodium lauryl sulfate, sodium N-coco, N-methyl taurate, sodium N-lauroyl sarcosine, or a compatible dental detergent.

### Preservatives

A pharmaceutically acceptable preservative can include one or a mixture of preservatives, such as, for example, benzoic acid, sodium benzoate, methylparaben, propylparaben, sorbic acid and potassium sorbate. These preservative agents are generally present at levels ranging from about 0.01% w/w to about 2% w/w. The example preservative is sodium benzoate.

### EXAMPLES

### Example. Diphenhydramine HCl Low GI Pectin Gummy

Ingredients: Water, pectin, 1040.5 g Allulose; 558.0 g Trehalose, 372.0 g Isomaltulose, Mannitol, Sodium Citrate, gamma-cyclodextrin, Maltodextrin, 10.00 g Diphenhydramine HCl, Citric Acid, Malic Acid, 12 g CFR Title 21 Granular Orange Flavor, 6 g CFR Title 21 beta-Carotene Color.

In a first container was added the allulose, trehalose, isomaltulose, maltodextrin, and mannitol. The carbohydrates were combined and mixed until homogeneous. This was Mix 1. Mix 1 was added to a separate container and water was added. The mixture was heated to boiling to create a hot syrup solution. The malic and citric acids were added to the syrup. In a second container was added the Diphenhydramine HCl, cyclodextrin and sodium citrate. This was Mix 2. Water was added to a container and heated to 99 C. Mix 2 was added to the water with stirring. This solution was allowed to stir for the 30 minutes. In a third container was added the pectin. Mix 1 was added to the pectin and mixed until homogeneous. This was Mix 3. Mix 3 was added to the water solution of Mix 2 with stirring. The solution was allowed to stir for 5-10 minutes. The pectin mixture was then added to the hot syrup. The mixture was allowed to heat until Brix 82 at which time the flavor and color were added. The solution was heated to Brix 83 and then added to molds.

### Example. Low GI Diphenhydramine HCl Gummy

Ingredients: 75.0 g pectin, 1040.5 g Allulose, 558.0 g Trehalose, 372.0 g Isomaltulose, Maltodextrin, 3Mannitol, Sodium Citrate; beta-cyclodextrin, 25.00 g Diphenhydramine HCl, Citric Acid, Malic Acid, 12 g CFR Title 21 Granular Cherry Color, CFR Title 21 Watermelon Red Color.

In a separate container was added allulose, trehalose, isomaltulose, gamma-cyclodextrin, maltodextrin and mannitol. They were combined and mixed until homogeneous. This was Mix 1. Mix 1 was added to a separate container and water was added. The mixture was heated and begins to boil to create a hot syrup solution. The malic and citric acids were added to the syrup. In a separate container was added the diphenhydramine HCl, beta-cyclodextrin and sodium citrate. This was Mix 2. Water was added to a container and heated to 99° C. Mix 2 was added to the water with stirring. This solution was allowed to stir for the 30 minutes. In a separate container was added the pectin. 300 g from Mix 1 was added to the pectin and mixed until homogeneous. This was Mix 3. Mix 3 was added to the water solution of Mix 2 with stirring. The solution was allowed to stir for 5-10 minutes. The pectin mixture was then added to the hot syrup. The mixture was allowed to heat until Brix 82 at which time the flavor and color were added. The solution was heated to Brix 83 and then added to molds.

### Example. Diphenhydramine HCl Sugar Free Pectin Gummy

Ingredients: pectin, 1040.5 g Sorbitol, 930.5 g Xylitol, Mannitol, Sodium Citrate, beta-cyclodextrin, Maltodextrin, 10.00 g Diphenhydramine HCl, Citric Acid, Malic Acid, 12 g CFR Title 21 Granular Orange Flavor, 6 g CFR Title 21 beta-Carotene Orange Color.

In a separate container was added the sorbitol, maltodextrin, cyclodextrin, xylitol and mannitol. These were combined and mixed until homogeneous. This was Mix 1. Mix 1 was added to a separate container and water was added. The mixture was heated and begins to boil to create a hot syrup solution. The malic and citric acids were added to the syrup. In a separate container was added the diphenhydramine HCl and sodium citrate. This was Mix 2. Water was added to a container and heated to 99° C. Mix 2 was added to the water with stirring. This solution was allowed to stir for 30 minutes. In a separate container was added the pectin. 300 g from Mix 1 was added to the pectin and mixed until homogeneous. This was Mix 3. Mix 3 was added to the water solution of Mix 2 with stirring. The solution was allowed to stir for 5-10 minutes. The pectin mixture was then added to the hot syrup. The mixture was allowed to heat until Brix 82 at which time the flavor and color were added. The solution was heated to Brix 83 and then added to molds.

### Example. Sugar Free Diphenhydramine HCl Gummy

Ingredients: pectin, 1200 g Maltitol, Isomalt, Maltodextrin, Mannitol, Sodium Citrate; beta-cyclodextrin, 10.00 g Diphenhydramine HCl, Citric Acid, Malic Acid, 12 g CFR Title 21 Granular Grape Flavor, 6 g CFR Title 21 Black Carrot Color.

In a separate container was added the maltitol, cyclodextrin, isomalt, maltodextrin and mannitol. These were combined and mixed until homogeneous. This was Mix 1. Mix 1 was added to a separate container and water was added. The mixture was heated and begins to boil to create a hot syrup solution. The malic and citric acids were added to the syrup. In a separate container was added diphenhydramine HCl and sodium citrate. This was Mix 2. water was added to a container and heated to 99° C. Mix 2 was added to the water with stirring. This solution was allowed to stir for the 30 minutes. In a separate container was added the pectin. 300 g from Mix 1 was added to the pectin and mixed until homogeneous. This was Mix 3. Mix 3 was added to the water solution of Mix 2 with stirring. The solution was allowed to stir for 5-10 minutes. The pectin mixture was then added to the hot syrup. The mixture was allowed to heat until Brix 82 at which time the flavor and color were added. The solution was heated to Brix 83 and then added to molds.

### Example. Sugar Free Diphenhydramine HCl Gummy

Ingredients: 75.0 g pectin, Maltitol, Isomalt, Maltodextrin, 30.0 g Mannitol, Sodium Citrate, 80.0 g beta-cyclodextrin, 25.00 g Diphenhydramine HCl, 4 g Citric Acid, Malic Acid, 12 g CFR Title 21 Granular Cherry Color, 6 g CFR Title 21 Watermelon Red Color.

In a separate container was added the maltitol, cyclodextrin, isomalt, maltodextrin and mannitol. The three were combined and mixed until homogeneous. This was Mix 1. Mix 1 was added to a separate container and water was added. The mixture was heated and begins to boil to create a hot syrup solution. The malic and citric acids were added to the syrup. In a separate container was added diphenhydramine HCl and sodium citrate. This was Mix 2. Water was added to a container and heated to 99° C. Mix 2 was added to the water with stirring. This solution was allowed to stir for the 30 minutes. In a separate container was added the pectin. 300 g from Mix 1 was added to the pectin and mixed until homogeneous. This was Mix 3. Mix 3 was added to the water solution of Mix 2 with stirring. The solution was allowed to stir for 5-10 minutes. The pectin mixture was then added to the hot syrup. The mixture was allowed to heat until Brix 82 at which time the flavor and color were added. The solution was heated to Brix 83 and then added to molds.

### Example: Sugar Free Diphenhydramine Pectin Gummy

Ingredients: pectin, Maltitol, Isomalt, 200 g Maltodextrin, 3 g Sodium Citrate, beta-Cylcodextrin, 30.0 g Mannitol, 12 g Cherry Flavor, and 11.6 g Diphenhydramine HCl, 8 g Citric Acid, and 1.5 g red color In a first container was added the pectin, a portion of isomalt, maltitol and the sodium citrate. The components were mixed until homogeneous. This was Mix 1. In a second container was added the remaining isomalt and maltitol, maltodextrin and mannitol. The components were mixed until homogeneous. This was Mix 2. In a third container was added the citric acid solution, red color, and cherry flavor. All ingredients were mixed together and warmed to 175° F. until all ingredients were dissolved. This was Mix 3. To a first reaction container was added the diphenydramine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 12.5 mg diphenhydramine.

### Example. Loratadine Low GI Pectin Gummy

Ingredients: Pectin, 1000 g Maltitol, Tehalose, Isomaltulose, 1.5 g Sodium Citrate, beta-Cyclodextrin, Mannitol, Maltodextrin, CFR 21 Orange Oil, 10 g Loratadine, 50% Citric Acid solution (in 50% glycerol/water), CFR 21 beta-Carotene Orange Color

In a first container was added the pectin, 200 g of maltitol, loratadine, and sodium citrate. The components were mixed until homogeneous. This was Mix 1. Water was heated to 200° F. in a container. To the hot water was added Mix 1. The mixture was stirred until the pectin fully swelled and dispersed. The solution was brought to a gentle boil. In a second container was added the remaining maltitol, trehalose, isomaltulose beta-cyclodextrin, and mannitol. The components were mixed until homogeneous. This was Mix 2. Mix 2 was added to a container followed by hot water. The container was heated until the contents were brought to a boil. In a separate container was added the citric acid solution, orange color, and blood orange flavor. All was mixed and warmed to 175° F. until all was dissolved. This was Mix 3. To the boiling Mix 1 solution was added Mix 2 with stirring. The mixture was heated to Brix 82 at which time Mix 3 was added dropwise with stirring. The gummy syrup was then added to silicone molds. The gummy pieces were removed from the molds to yield products containing 10 mg loratadine per piece.

### Example. Loratadine Sugar Free Pectin Gummy

Ingredients: Pectin, Isomalt, 1.5 g Sodium Citrate, beta-Cyclodextrin, Mannitol, 1005 g Maltitol, 200 g Maltodextrin, CFR 21 Orange Oil, 10 g Loratadine, 50% Citric Acid solution (in 50% glycerol/water), CFR 21 beta-Carotene Orange Color

In a separate container was added the pectin, 200 g of isomalt, loratadine, and sodium citrate. The components were mixed until homogeneous. This was Mix 1. Water was heated to 200° F. in a container. To the hot water was added Mix 1. The mixture was stirred until the pectin fully swells and disperses which takes roughly 3-5 minutes. The solution was brought to a gentle boil. In a separate container was added the remaining isomalt, maltitol, beta-cyclodextrin, and mannitol. The components were mixed until homogeneous. This was Mix 2. Mix 2 was added to a container followed by hot water. The container was heated until the contents were brought to a boil. In a separate container was added the citric acid solution, orange color, and blood orange flavor. All was mixed and warmed to 175° F. until all was dissolved. This was Mix 3. To the boiling Mix 1 solution was added Mix 2 with stirring. The mixture was heated to Brix 82 at which time Mix 3 was added dropwise with stirring. The gummy syrup was then added to silicone molds. The gummy pieces were removed from the molds to yield products containing 10 mg loratadine per piece.

### Example: Sugar Free Loratadine Pectin Gummy w/0.033% Sucralose

Ingredients: pectin, g Maltitol, Isomalt, Polydextrose Soluble Fiber, 4.5 g Sodium Citrate, beta-Cylcodextrin, 90.0 g Mannitol, 12 g Watermelon Flavor, and 9.45 g Loratadine, 5.01 g Citric Acid, and 1.8 g red color, 5 g Glycerol, 0.99 g Sucralose.

In a first container was added the pectin, a portion of isomalt, maltitol, sucralose and the sodium citrate. The components were mixed until homogeneous. This was Mix 1. In a second container was added the remaining isomalt and maltitol, polydextrose and mannitol. The components were mixed until homogeneous. This was Mix 2. In a third container was added the citric acid solution, red color, and watermelon flavor. All ingredients were mixed together and warmed to 175° F. until all ingredients were dissolved. This was Mix 3. To a first reaction container was added the loratadine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg loratadine.

### Example: Sugar Free Loratadine Pectin Gummy w/0.033% Sucralose

Ingredients: pectin, 1125 g Maltitol, Isomalt, 450 g Fructooligiosaccarides, 4.5 g Sodium Citrate, beta-Cylcodextrin, 90.0 g Mannitol, 12 g Watermelon Flavor, and 9.45 g Loratadine, 5.01 g Citric Acid, and 1.8 g red color, 15 g Glycerol, 0.99 g Sucralose.

In a first container was added the pectin, a portion of isomalt, maltitol, sucralose and the sodium citrate. The components were mixed until homogeneous. This was Mix 1. In a second container was added the remaining isomalt and maltitol, polydextrose and mannitol. The components were mixed until homogeneous. This was Mix 2. In a third container was added the citric acid solution, red color, and watermelon flavor. All ingredients were mixed together and warmed to 175° F. until all ingredients were dissolved. This was Mix 3. To a first reaction container was added the loratadine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg loratadine.

### Example. Cetirizine Sugar Free Gelatin Gummy

Ingredients: Sorbitol, Mannitol, 1.3 g Cetirizine HCl, Water, Potassium Sorbate, Sodium Benzoate, Gelatin, Isomalt, Taurine

The gelatin, mannitol, cetirizine and taurine were shifted together until homogenous. The dry component mixture was added to 116.0 g of water rapidly with rapid stirring. The dry Mix quickly absorbed the water and became a rubbery mass. The rubbery mass was added to a zip lock bag and heated at 160° F. until free of foam and a clear yellow. Water was added to a container and heated to the boiling point. To the boiling water was added potassium sorbate and sodium benzoate, which dissolved into the boiling water. To the boiling solution was added sorbitol and isomalt. The solution was then brought to a boil and sucrose was added. The solution was heated until 248° F. was reached and 108 g of water removed. The solution was then cooled to 200° F. and the gelatin solution was slowly added with stirring. The mixture was stirred until homogenous. The solution was then added to silicone molds and the molds were placed in the refrigerator for 90 minutes. The gummy pieces were removed from the molds to yield products containing roughly 11 mg cetirizine per piece.

### Example. Cetirizine Gelatin Sugar Free Gummy

Ingredients: Sorbitol, Mannitol, 1.3 g Cetirizine HCl, Water, Potassium Sorbate, Sodium Benzoate, Gelatin, Isomalt, alpha-cyclodextrin, Taurine

The gelatin, mannitol, alpha-cyclodextrin, cetirizine and taurine were shifted together until homogenous. The dry component mixture was added to 116.0 g of water rapidly with rapid stirring. The dry Mix quickly absorbed the water and became a rubbery mass. The rubbery mass was added to a zip lock bag and heated at 160° F. until free of foam and a clear yellow. Water was added to a container and heated to the boiling point. To the boiling water was added potassium sorbate and sodium benzoate, which dissolved into the boiling water. To the boiling solution was added sorbitol and isomalt. The solution was then brought to a boil and sucrose was added. The solution was heated until 248° F. was reached and 108 g of water removed. The solution was then cooled to 200° F. and the gelatin solution was slowly added with stirring. The mixture was stirred until homogenous. The solution was then added to silicone molds and the molds were placed in the refrigerator for 90 minutes. The gummy pieces were removed from the molds to yield products containing roughly 11 mg cetirizine per piece.

### Example. Cetirizine Sugar Free Gummy

Ingredients: Isomalt, Mannitol, 1.3 g Cetirizine HCl, Water, Potassium Sorbate, Sodium Benzoate, Gelatin, Maltitol, alpha-cyclodextrin, Taurine, Orange Flavor, Carotene color.

The gelatin, mannitol, alpha-cyclodextrin, cetirizine and taurine were shifted together until homogenous. The dry component mixture was added to 116.0 g of water rapidly with rapid stirring. The dry Mix quickly absorbed the water and became a rubbery mass. The rubbery mass was added to a zip lock bag and heated at 160° F. until free of foam and a clear yellow.

Water was added to a container and heated to the boiling point. To the boiling water was added potassium sorbate and sodium benzoate which dissolved into the boiling water. To the boiling solution was added maltitol and isomalt. The solution was then brought to a boil and sucrose was added. The solution was heated until 248° F. was reached and 108 g of water removed. The solution was then cooled to 200° F. and the gelatin solution was slowly added with stirring. The mixture was stirred until homogenous at which time orange flavor and carotene color were added. The solution was then added to silicone molds and the molds were placed in the refrigerator for 90 minutes. The gummy pieces were removed from the molds to yield products containing roughly 11 mg cetirizine per piece.

### Example. Cetirizine Gelatin Sugar Reduced Ginger Gummy

Ingredients: Maltitol Syrup, Ginger Juice Concentrate, Sorbitol, Cetirizine Hydrochloride, Gelatin, Mannitol, Taurine, Sodium Benzoate, Potassium Sorbate, alpha-Cyclodextrin

The gelatin, cetirizine, alpha-cyclodextrin and mannitol were shifted together until homogenous. The dry component mixture was added to water with dissolved potassium sorbate and sodium benzoate rapidly with rapid stirring. The dry Mix quickly absorbed the water and became a rubbery mass. The rubbery mass was added to a zip lock bag and heated at 160° F. until free of foam and a clear yellow solution. The ginger concentrate was added to a container with sorbitol and maltitol syrup. The components were heated until reflux until a Brix level of 87.5 was reached. The components were cooled to 200° F. and the gelatin Mix was added with stirring. The final Brix was 84. The solution was then added to silicone molds and the molds were placed in the refrigerator for 90 minutes. The gummy pieces were removed from the molds to yield products containing 11 mg cetirizine per piece.

### Example. Cetirizine Sugar Free Pectin Gummy with Oil

Ingredients: Pectin (Herbstreith & Fox), Sorbitol, Sodium Citrate, Sucrose, Clusterdextrin, Mannitol, Coconut Oil, (Boling) Maltitol Syrup, Blood Orange Extract, Cetirizine HCl, 50% Citric Acid solution (in 50% glycerol/water), glycerol, orange color

In a separate container was added the pectin, 100 g of sorbitol, cetirizine. and the sodium citrate. The components were mixed until homogeneous. This was Mix 1. In a separate container was added the remaining sucrose, clusterdextrin, and mannitol. The components were mixed until homogeneous. This was Mix 2. In a separate container was added the citric acid solution, orange color, and blood orange flavor. All was mixed and warmed to 175° F. until all was dissolved. This was Mix 3.

Water was heated to 200° F. in a saucepan. To the hot water was added Mix 1. The mixture was stirred until the pectin fully swells and disperses which takes roughly 3-5 minutes. The solution was brought to a gentle boil. To the boiling Mix 1 solution was added Mix 2 with stirring. Coconut oil was then added dropwise to the boiling mixture with stirring. The boiling glucose syrup was then added to the boiling pectin/sugar alcohol/oil mix. The mixture was heated to Brix 82 at which time Mix 3 was added dropwise with stirring. The modling mix was then added to silicone molds. The gummy pieces were removed from the molds to yield products containing 11 mg cetirizine per piece.

### Example. Cetirizine Sugar Free Pectin Gummy

Ingredients: Pectin (Herbstreith & Fox), Sorbitol, Sodium Citrate, Sucrose, Cluster dextrin, Mannitol, (Boling) Maltitol Syrup, Blood Orange Extract, Cetirizine HCl, 50% Citric Acid solution (in 50% glycerol/water), glycerol, orange color

In a separate container was added the pectin, 100 g of sorbitol, cetirizine. and the sodium citrate. The components were mixed until homogeneous. This was Mix 1. In a separate container was added the remaining sucrose, clusterdextrin, and mannitol. The components were mixed until homogeneous. This was Mix 2. In a separate container was added the citric acid solution, orange color, and blood orange flavor. All was mixed and warmed to 175° F. until all was dissolved. This was Mix 3. Water was heated to 200° F. in a saucepan. To the hot water was added Mix 1. The mixture was stirred until the pectin fully swells and disperses which takes roughly 3-5 minutes. The solution was brought to a gentle boil. To the boiling Mix 1 solution was added Mix 2 with stirring. The boiling glucose syrup was then added to the boiling pectin/sugar alcohol/oil mix. The mixture was heated to Brix 82 at which time Mix 3 was added dropwise with stirring. The modling mix was then added to silicone molds. The gummy pieces were removed from the molds to yield products containing 11 mg cetirizine per piece.

### Example. Agar Cetirizine Sugar Free Gummy

Ingredients: Ticagel Natural GC-581 B, Isomalt, Sorbitol, Mannitol, Cetirizine HCl, Citric Acid 50% in Water, orange color, Orange Natural Flavor, Glycerin

While stirring, add the Ticagel to water heated to 175° F. and Mix until free of lumps. This takes roughly 10 minutes. Isomalt dissolved into water and the syrup was prewarmed to 175° F. The syrup was combined with the agar Mix with stirring. Added the sorbitol with mixing for two minutes. Heat until a Brix of 80 was reached. The 50% citric acid, color, orange natural flavor, cetirizine, and glycerol were combined until homogenous. This mixture was then added to the batter. The batter was then deposited into silicone molds and cured at 98° F. and 18% relative humidity for 24 hours. The gummy pieces were removed from the molds to yield products containing 11 mg cetirizine per piece.

### Example. Kappa Carrageenan Cetirizine Sugar Free Gummy

Ingredients: Kappa Carrageenan, Potassium Citrate, Isomalt, Coconut oil, Sucrose, Maltitol Syrup, Cetirizine HCl, Mannitol, Blood Orange Extract

In a separate container was combined the kappa carrageenan, 100 g isomalt, and potassium citrate. These components were mixed to water heated to 200° F. with stirring until lump free. This took approximately 10 minutes. In a separate container was added the maltitol syrup with 220 g of isomalt and the mannitol. These were heated together until boiling. The mixture was heated until Brix 88 was reached. The mixture was cooled to 210° F. and the above solution of kappa carrageenan was slowly added with stirring. The cetirizine, blood orange extract, and 5 g water were combined and added drop wise to the kappa/sugar mixture. The resulting modling mix was then added to silicone molds. The gummy pieces were removed from the molds to yield products containing 11 mg cetirizine per piece.

### Example: Sugar Free Cetirizine Pectin Gummy

Ingredients: pectin, 372.0 g Sorbitol, Isomalt, 1.5 g Sodium Citrate, beta-Cylcodextrin, Mannitol, 6.5 g Orange Flavor, and 2.70 g Cetirizine HCl, 20.0 g 50% Citric Acid solution (in 50% glycerol/water), and 0.6 g orange color

In a first container was added the pectin, a portion of sorbitol, a portion of isomalt and the sodium citrate. The components were mixed until homogeneous. This was Mix 1. In a second container was added the remaining sorbitol, isomalt, and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, Allulose, 240.0 g Maltitol, Inulin, 4.5 g Sodium Citrate, beta-Cylcodextrin, 25.0 g Mannitol, 6.5 g Orange Flavor, and 2.70 g Cetirizine HCl, 5 g Citric Acid, and 0.6 g orange color

In a first container was added the pectin, a portion of allulose, a portion of maltitol and the sodium citrate. The components were mixed until homogeneous. This was Mix 1. In a second container was added the remaining allulose, maltitol, inulin and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, Maltitol, 200 g Maltodextrin, 4.5 g Sodium Citrate, beta-Cylcodextrin, 25.0 g Mannitol, 6.5 g Orange Flavor, and 2.70 g Cetirizine HCl, 10 g Citric Acid, and 0.6 g orange color

In a first container was added the pectin, a portion of maltitol and the sodium citrate. The components were mixed until homogeneous. This was Mix 1. In a second container was added the remaining maltitol, maltodextrin and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, 357.0 g Maltitol, Maltodextrin, 4.5 g Sodium Citrate, beta-Cylcodextrin, Mannitol, 6.5 g Orange Flavor, and 2.70 g Cetirizine HCl, 10 g Citric Acid, and 0.6 g orange color

In a first container was added the pectin, a portion of maltitol and the sodium citrate. The components were mixed until homogeneous. This was Mix 1. In a second container was added the remaining maltitol, maltodextrin and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, 357.0 g Maltitol, Maltodextrin, 4.5 g Sodium Citrate, beta-Cylcodextrin, 25.0 g Mannitol, 6.5 g Orange Flavor, and 2.70 g Cetirizine HCl, Citric Acid, and 0.6 g orange color

In a first container was added the pectin, a portion of maltitol and the sodium citrate. The components were mixed until homogeneous. This was Mix 1. In a second container was added the remaining maltitol, maltodextrin and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, 1150.55 g Maltitol, Isomalt, 185 g Maltodextrin, Sodium Citrate, beta-Cylcodextrin, Mannitol, 12 g Orange Flavor, and 11 g Cetirizine HCl, 8 g Citric Acid, and 1 gram orange color

In a first container was added the pectin, a portion of isomalt, maltitol and the sodium citrate. The components were mixed until homogeneous. This was Mix 1. In a second container was added the remaining isomalt and maltitol, maltodextrin and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

### Example: Sugar Free Cetirizine Pectin Gummy

Ingredients: pectin, 1150.55 g Maltitol, 820.45 g Isomalt, Polydextrose Soluble Fiber, 3 g Sodium Citrate, beta-Cylcodextrin, 30.0 g Mannitol, Orange Flavor, and 11 g Cetirizine HCl, 8 g Citric Acid, and orange color

In a first container was added the pectin, a portion of isomalt, maltitol and the sodium citrate. The components were mixed until homogeneous. This was Mix 1. In a second container was added the remaining isomalt and maltitol, polydextrose and mannitol. The components were mixed until homogeneous. This was Mix 2. In a third container was added the citric acid solution, orange color, and orange flavor. All ingredients were mixed together and warmed to 175° F. until all ingredients were dissolved. This was Mix 3. To a first reaction container was added the cetirizine with beta-cyclodextrin. Water was heated to 200° F. and were then added to the reaction container. To the hot water was added Mix 1 with stirring. The mixture was stirred until the pectin fully swelled and dispersed. The solution was brought to a light boil. To a second reaction container, was added Mix 2, followed by water. The components were mixed and brought to a boil. Then, the dispersed pectin Mix was slowly added with mixing. The mixture was heated to 82 Brix, at which time Mix 3 was added with stirring. The modling mix was then added to gummy molds to create sugar free cetirizine gummy pieces each containing 10 mg cetirizine.

What is claimed is:

1. An antihistamine semi-solid chewable gel composition, comprising,
an active pharmaceutical ingredient (API) composition comprising an antihistamine,
a gelling composition in a sufficient amount to provide a cohesive gelled product,
a binding composition comprising a sugar, a sugar alcohol, or a combination thereof, wherein the sugar comprises L-fructose, L-glucose, L-galactose, allulose, sorbose, tagatose, D-maltose (1,4-diglucose), trehalose, isomaltulose, raffinose or a combination thereof, and
a water-soluble polymer stabilizer, wherein the polymer stabilizer comprises a polymer of monosaccharide monomers selected from glucose, mannose, galactose, arabinose, rhamnose, xylose, galacturonate, glucuronate, N-acetylglucosamine, or a combination thereof, and wherein the polymer comprises from about 5 to about 500 monosaccharide monomers,
wherein the semi-solid chewable gel composition is substantially free of glucose, sucrose, and fructose.

2. The antihistamine semi-solid chewable gel composition of claim 1, wherein the binding composition comprises mannitol, maltitol, isomalt, erythritol, sorbitol, xylitol, tagatose, allulose, sorbose, isomaltulouse, trehalose, mannose, maltose, ribose, xylose, their acid forms or a combination thereof.

3. The antihistamine semi-solid chewable gel composition of claim 1, wherein the polymer of monosaccharide monomers comprise glucose monomer or mannose monomer linked through glyosidic bonds substantially free of alpha-1,4-glycosidic bond.

4. The anti-histamine semi-solid chewable gel composition of claim 1, wherein the polymer stabilizer comprises polydextrose soluble fiber, resistant starch, cellulose, maltodextrin, resistant maltodextrin, beta-glycan, inulin, oligofructose, fruicto-oligosaccharide, mannan-oligosaccahride, galacto-oligosaccahride, galactomannan oligomers, oligomers of ribose, xylose, arabinose, rhamnose, or a combination thereof.

5. The anti-histamine semi-solid chewable gel composition of claim 1, wherein the gelling composition comprises gelatin, starch, pectin, gellan gum, guar gum, alginin, gum Arabic, carrageenan, guar, agar, carboxymethylcellulose, hydroxyethylcellulose, sago, alginate, locust bean gum, xanthan gum, or derivatives thereof.

6. The antihistamine semi-solid chewable gel composition of claim 1, further comprising a complexing composition, wherein the complexing composition comprises cyclodextrin, an amide, a nucleotide, resistant starch, a peptide, or a combination thereof.

7. The antihistamine semi-solid chewable gel composition of claim 6, wherein the amide comprises N-acetyl glucosamine, N-acetyl galactosamine, or a combination thereof.

8. The antihistamine semi-solid chewable gel composition of claim 1, further comprising an herbal composition, an antioxidant composition, a vitamin composition, a mineral composition, an amino acid composition, a probiotics composition, or a prebiotics composition.

9. The semi-solid chewable gel composition of claim 1, further comprising an additive selected from a sweetener, a food acid, a flavoring agent, a coloring agent, a humectant, a bulking agent, a fatty acid, a triglyceride, a plasticizer, an emulsifier, a thickener, a preservative, or a mixture thereof.

10. The semi-solid chewable gel composition of claim 1, wherein the anti-histamine comprises acrivastine, azelastine, diphenhydramine, bilastine, bromodiphenhydramine, brompheniramine, buclizine, carbinoxamine, cetirizine, chlorodiphenhydramine, chlorphenamine, clemastine, cyclizine, cyproheptadine, dexbrompheniramine, dexchlorpheniramien, dimenhydrinate, dimetindene, doxylamine, ebastine, embramine, fexofenadine, hydroxyzine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, or a salt or derivative thereof.

11. The antihistamine semi-solid chewable gel composition of claim 1, the API composition further comprises a H2 receptor blocker, a decongestant, a corticosteroid, a Leukotriene inhibitor, or a combination thereof.

12. The anti-histamine semi-solid chewable gel composition of claim 11, wherein the decongestant comprises pseudoephedrine, oxymetazoline, tetrahydrozoline, a derivative or a combination thereof, wherein the Corticosteroid comprises budesonide, fluticasone furoate, fluticasone propionate, mometasone, triamcinolone, beclomethasone, ciclesonide, fluorometholone, loteprednol, prednisolone, prednisone, methylprednisolone, a derivative or a combination thereof, or wherein the leukotriene inhibitor comprises montelukast, a derivative thereof.

13. The semi-solid chewable gel composition of claim 1, comprising the antihistamine at a concentration not less than about 0.05% w/w.

14. The semi-solid chewable gel composition of claim 1, wherein the antihistamine comprises cetirizine, levocetirizine, diphenhydramine, loratadine, or fexofenadine, and wherein the complexing composition comprises polyamide, cluster dextrin, cyclodextrin, or a combination thereof.

15. The antihistamine semi-solid chewable gel composition of claim 1, comprising at least 2% w/w of the polymer stabilizer.

16. The antihistamine semi-solid chewable gel composition of claim 1, wherein the weight ratio of the binding composition and the polymer stabilizer is from about 10:1 to 20:1.

17. The antihistamine semi-solid chewable gel composition of claim 1, further comprising a coating composition, wherein the coating composition comprises isomalt, allulose, tagatose, xylitol, erythritol, sorbitol, mannitol, or a combination thereof

18. The antihistamine semi-solid chewable gel composition of claim 29, wherein the binding composition comprises maltitol and at least one of allulose, xylitol, erythritol, maltitol, sorbitol, or mannitol, wherein the sanding composition comprises maltitol.

19. A method of making the antihistamine semi-solid chewable gel composition of claim 6, comprising,
dividing the binding composition into a first binding portion and a second binding portion
combining a first mixture and water and heating to at a first elevated temperature to provide a first solution, wherein the first mixture comprising the first binding portion, the water-soluble polymer stabilizer,
combining a second mixture and water at a second elevated temperature to provide a second solution, wherein the second mixture comprises the gelling composition, the second binding portion, the complexing composition, the API composition, and wherein the second binding portion equals to at least twice the mass of the gelling composition,
combining the second solution and the first solution at a third elevated temperature to provide a third solution, wherein the third solution has a Brix number from about 80 to about 85,
adjusting pH of the third solution with a buffer salt to from about 3 to about 7.

20. The method of claim 19, further comprising the step of adding a coloring agent, a flavoring agent, or a combination thereof into the third solution to provide a molding mix having a Brix from about 78 to about 86.

21. The method of claim 20, further comprising the step of adding the molding mix to a preformed shaped cavity.

22. The method of claim 18, wherein the first, second and third elevated temperature is independently from about 175° F. to about 275° F.

23. A method for treating an allergy symptom in a subject, comprising administering to the subject an effective amount of the semi-solid chewable gel composition of claim 1, wherein the allergy symptom is associated with seasonal allergic rhinitis, perennial allergic rhinitis, or chronic urticaria.

24. The method of claim 23, wherein the allergy symptom comprises allergic rhinitis, sneezing, rhinorrhea, postnasal discharge, nasal pruritus, ocular pruritus, tearing, itchy eyes, urticaria, or a combination thereof.
